(19) United States
(12) Patent Application Publication
(10) Pub. No.: US 2026/0340840 A1
(43) Pub. Date: Oct. 29, 2026

(54) LENS FRAGMENTATION TRACKING

(71) Applicant: ALCON INC., Fribourg (CH)

(72) Inventors: Zhuoran WU, Fort Worth, TX (US); Lu YIN, Fort Worth, TX (US); Vignesh SURESH, Fort Worth, TX (US); Sinchan BHATTACHARYA, Fort Worth, TX (US); Aakash NAGARAJAN, Fort Worth, TX (US); Nikhil SCARIA, Fort Worth, TX (US)

(21) Appl. No.: 19/650,272

(22) Filed: Apr. 16, 2026

Related U.S. Application Data
(60) Provisional application No. 63/794,984, filed on Apr. 25, 2025.

(51) Int. Cl. G06T 7/246; A61B 90/00; A61F 9/007; G06T 7/12; G16H 30/20; A61B 17/00; G06V 10/25; G06V 10/82; G06V 20/40; G06V 40/18

(52) U.S. Cl. CPC G06T 7/246; A61B 90/361; A61F 9/00745; G06T 7/12; G16H 30/20; A61B 2017/00199; A61B 2217/005; G06T 2207/10016; G06T 2207/20084; G06T 2207/30041; G06V 10/25; G06V 10/82; G06V 20/40; G06V 40/18; G06V 2201/03

(57) ABSTRACT

A method of tracking lens material may include obtaining video data of an eye during a medical procedure on the eye. The method may also include identifying, during the medical procedure, multiple lens fragments in a pupil region of the eye by providing the video data to an artificial intelligence model. In these and other embodiments, the lens fragments may result from fragmentation of a lens of the eye during the medical procedure. The method may also include analyzing the video data during the medical procedure to detect whether any of the identified plurality of lens fragments move outside a boundary of the pupil region and in response to detecting one or more lens fragments moving outside the boundary of the pupil region, generating an indication that the one or more lens fragments moved outside the boundary of the pupil region.

### CROSS-REFERENCE TO RELATED APPLICATION

This application claims priority to U.S. Provisional Application No. 63/794,984, filed on Apr. 25, 2025, which is incorporated herein in its entirety.

### FIELD

The embodiments discussed herein relate to lens fragmentation tracking.

### BACKGROUND

Cataract surgery is a procedure in which the natural crystalline lens of the eye is removed and replaced with an artificial intraocular lens. For example, the procedure involves emulsification of the lens into fragments, such as soft lens material fragments, followed by aspiration of those fragments from the capsular bag. A surgeon may encounter residual lens fragments that migrate peripherally within the eye, toward or beneath the iris, where the fragments become invisible under standard surgical microscope visualization. Retained lens material, such as fragments that remain in the eye following the procedure, may result in elevated intraocular pressure, chronic inflammation, and other postoperative complications.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of at least one embodiment, a method of tracking lens material may include obtaining video data of an eye during a medical procedure on the eye. The method may also include identifying, during the medical procedure, multiple lens fragments in a pupil region of the eye by providing the video data to an artificial intelligence model. In these and other embodiments, the lens fragments may result from fragmentation of a lens of the eye during the medical procedure. The method may also include analyzing the video data during the medical procedure to detect whether any of the identified plurality of lens fragments move outside a boundary of the pupil region and in response to detecting one or more lens fragments moving outside the boundary of the pupil region, generating an indication that the one or more lens fragments moved outside the boundary of the pupil region.

The object and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1A illustrates an example environment for tracking lens material;

FIG. 1B illustrates an example eye during tracking of lens material;

FIG. 2 illustrates a flowchart of an example method of tracking lens material;

FIG. 3 illustrates a flowchart of another example method of tracking lens material;

FIG. 4 is a block diagram of an example computing system for generative artificial intelligence (AI); and

FIG. 5 is a block diagram of an example computing device, all in accordance with at least some embodiments of the present disclosure.

### DETAILED DESCRIPTION

During cataract surgery, a surgeon removes the natural crystalline lens of the eye, which is located behind the pupil, by breaking the lens into small fragments and sucking those fragments out of the eye. The fragments, referred to sometimes as soft lens material fragments but referred to in this disclosure as lens fragments, may vary in size and in some instances may move rapidly within the eye. As a result, the lens fragments may move from the pupil to beneath the iris, or colored region of the eye. Beneath the iris, the lens fragments may be difficult to locate.

In some circumstances, when lens fragments are not removed from the eye, postoperative inflammation, elevated intraocular pressure, and/or other complications may arise. The difficulty of visually tracking small, fast-moving, semi-transparent lens fragments—particularly those that slip beneath the iris—represents a challenge in eye surgery that existing visualization systems do not adequately address.

The present disclosure relates generally to systems and methods that may detect, track, and generate alerts regarding lens fragments during a medical procedure on an eye. For example, video data of the eye may be obtained during the medical procedure and used to identify lens fragments in a pupil region of the eye. The identified lens fragments may be analyzed across successive frames of the video data to detect whether any of the identified lens fragments move outside a boundary of the pupil region. In response to detecting one or more lens fragments moving outside the boundary of the pupil region, an indication that the one or more lens fragments moved outside the boundary of the pupil region may be generated. As such, the present disclosure may assist a surgeon in confirming that all lens fragments have been removed from the eye.

In some embodiments, to be able to detect and track lens fragments that move outside the boundary of the pupil region, the video data of the eye may be provided to a processing system. The video data may be processed on a frame-by-frame basis, and lens fragments in the pupil region may be identified in the video data by providing the video data to an artificial intelligence model. The artificial intelligence model may segment each frame of the video data to produce masks that identify the lens fragments and anatomical structures of the eye, including the pupil and the iris. Each lens fragment may be tracked across successive frames. For each frame, disappearance and a velocity of each of the lens fragments and a total volume of all the lens fragments may be analyzed to detect whether any of the identified lens fragments move outside the boundary of the pupil region. In response to detecting one or more lens fragments moving outside the boundary of the pupil region, an indication may be generated and presented on a display by overlaying the indication on the video data that is being presented to a surgeon. As a result, the systems and methods provided in this disclosure may provide a surgeon with real-time visual feedback regarding the location and movement of lens fragments that may have moved beneath the iris.

In some embodiments, the indication generated may include a visual indicator of an approximate location of the one or more lens fragments outside the boundary of the pupil region. The visual indicator may be overlaid on the video data presented on a display, providing the surgeon with spatially referenced information regarding the location of lens fragments that may have moved beneath the iris. The display may also present other graphical elements, such as highlighting lens fragments that are approaching the boundary of the pupil region to augment the surgeon's situational awareness during the medical procedure.

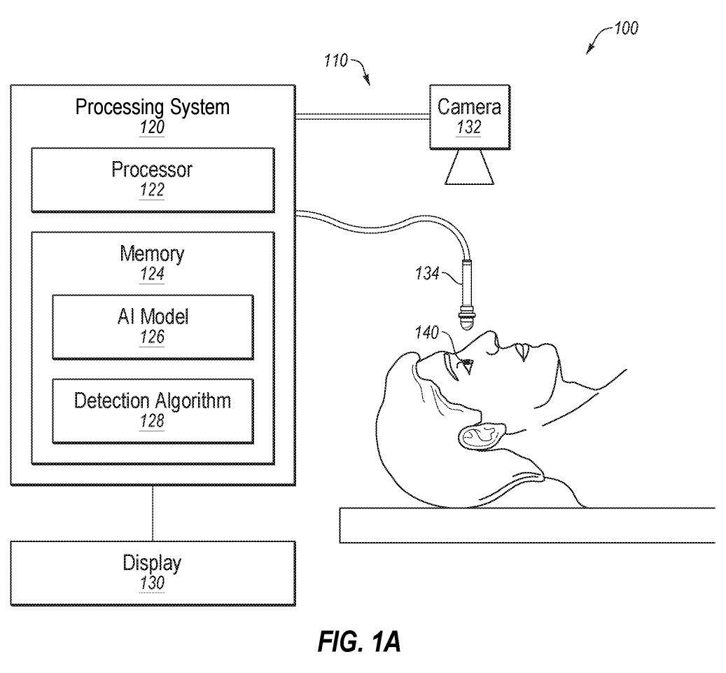

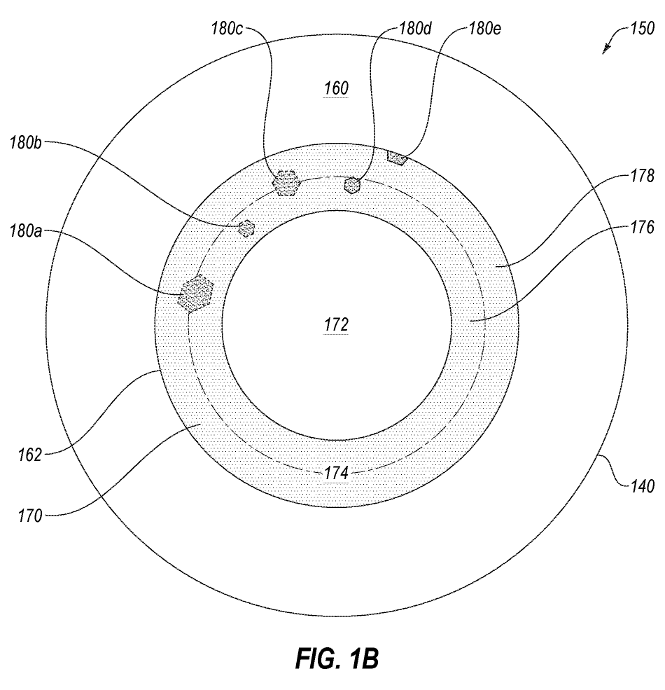

Turning to the figures, FIG. 1A illustrates an example environment 100 for tracking lens material during a medical procedure on an eye 140 of a patient. The environment 100 may be arranged in accordance with at least one embodiment described in the present disclosure. The environment 100 may include the eye 140 and a medical system 110 that includes a processing system 120, a display 130, a camera 132, and an aspiration probe 134.

In some embodiments, the medical system 110 may be configured to operate during various ophthalmic surgical procedures in which detection and tracking of particles, fragments, or debris within the eye 140 may be performed.

For example, in some embodiments, the medical system 110 may be configured to operate during a cataract surgery procedure. In some circumstances, cataract surgery may be performed to remove a natural crystalline lens of the eye 140. During the procedure, a surgeon may make one or more small incisions in the eye 140 to gain access to the crystalline lens. The aspiration probe 134 may be inserted through one of the incisions and may be configured to vibrate at ultrasonic frequencies to emulsify the crystalline lens into lens fragments. The aspiration probe 134 may also aspirate the lens fragments out of the eye 140 through a vacuum-driven suction mechanism. Following emulsification and aspiration of the lens fragments, an artificial intraocular lens may be implanted into the posterior lens capsule of the eye 140 to restore optical function.

In some embodiments, the lens fragments produced during the emulsification step may vary in size and may move rapidly within the eye 140. In these and other embodiments, the lens fragments may migrate from a pupil region toward an iris 160 of the eye 140 and may pass beneath the iris. When the lens fragments pass beneath the iris, the lens fragments may no longer be directly visible to the surgeon. In these and other embodiments, the medical system 110 may help track lens fragments to inform the surgeon about whether all, or nearly all, fragments have been removed from the eye 140.

For example, the medical system 110 may track the lens fragments and may provide an indication to the surgeon when a lens fragment approaches and/or passes beneath the iris. Without the tracking by the medical system 110, the surgeon may be unaware that a lens fragment is moving toward the iris and/or passed beneath the iris. Furthermore, the surgeon may not be able to see the lens fragments that are now beneath the iris. As a result, without the indication from the medical system 110, the surgeon may think all lens fragments have been removed and may not remove the lens fragment that is now beneath the iris. The lens fragment remaining beneath the iris may result in complications for the patient.

Based on the indications from the medical system 110, the surgeon may locate and aspirate lens fragments before the lens fragments move beneath the iris and/or perform additional aspiration and collect the lens fragments that moved beneath the iris. In some embodiments, the medical system 110 may also assist the surgeon in removing lens fragments that travel beneath the iris by generating spatially referenced information regarding the location of lens fragments that have moved beneath the iris.

In some embodiments, the medical system 110 may be configured to operate during other ophthalmic surgical procedures in which detection and tracking of particles, fragments, or debris within the eye 140 may be performed. For example, the medical system 110 may be configured to operate during a vitreoretinal surgical procedure in which the vitreous humor of the eye 140 may be partially or fully removed and residual tissue fragments or debris may migrate within the posterior segment of the eye 140. As another example, the medical system 110 may be configured to operate during a glaucoma surgical procedure in which tissue fragments may be generated and may require tracking to confirm complete removal from the eye 140. As yet another example, the medical system 110 may be configured to operate during a laser eye surgery procedure, such as a LASIK (Laser-Assisted In Situ Keratomileusis) procedure, in which ablated tissue particles may be generated within the surgical field of the eye 140 and may require monitoring to confirm that the particles do not migrate to unintended regions of the eye 140.

In some embodiments, the camera 132 may be configured to generate video data of the eye 140 during the medical procedure. In some embodiments, the camera 132 may be part of a digital ophthalmic surgical microscope system. For example, the camera 132 may be part of a digital visualization system configured to capture high-resolution digital video of the eye 140 during surgery. In some embodiments, the camera 132 may output video data that includes a continuous digital video stream that includes sequential video frames. The camera 132 may transmit the video data to the processing system 120 via a digital interface. In some embodiments, the camera 132 may capture the surgical field, including the cornea, the iris, the pupil, and any lens fragments present within the field of view.

In some embodiments, the aspiration probe 134 may be configured to aspirate a plurality of lens fragments resulting from fragmentation of the lens of the eye 140. In some embodiments, the aspiration probe 134 may be an irrigation and aspiration handpiece configured to remove residual lens fragments from the capsular bag of the eye 140 using vacuum-driven suction. Alternately or additionally, the aspiration probe 134 may also be configured to vibrate at ultrasonic frequencies to emulsify tissue into fragments, such as emulsifying a lens into lens fragments.

In some embodiments, the aspiration probe 134 may be inserted into the eye 140 through a small incision and may be repositioned by a surgeon throughout the medical procedure to aspirate lens fragments from different regions of the eye 140. In some embodiments, the aspiration probe 134 may be visible within the video data generated by the camera 132. In these and other embodiments, a location of the aspiration probe 134 within a boundary of a pupil region of the eye 140 may be used to define a portion of the pupil region within which lens fragments may be tracked.

The processing system 120 may include a processor 122 and a memory 124. In some embodiments, the processing system 120 may include any configuration of hardware, such as processors, servers, and database servers that may be networked together and configured to perform operations including detecting, tracking, and generating alerts regarding lens fragments during the medical procedure.

In some embodiments, the processing system 120 may be a workstation integrated with or connected to the camera 132. In some embodiments, the processing system 120 may be an embedded computing system within a surgical microscope platform.

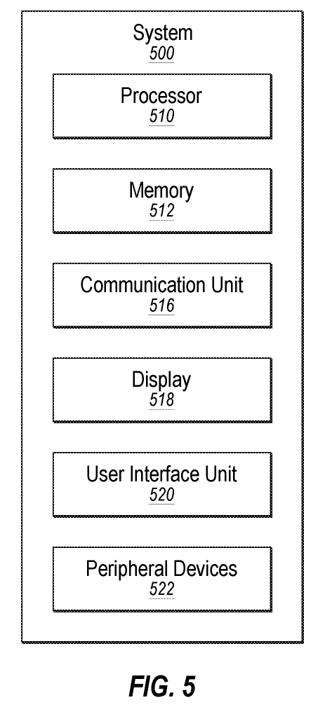

The processor 122 may include any suitable special-purpose or general-purpose computing entity or processing device including various computer hardware or software modules and may be configured to execute instructions stored on any applicable computer-readable storage media. For example, the processor 122 may include a GPU configured to perform accelerated deep learning model inference and a CPU configured to perform pipeline orchestration, tracking computations, and alert generation. An example processor is further described with respect to FIG. 5

The memory 124 may include computer-readable storage media or one or more computer-readable storage mediums for carrying or having computer-executable instructions or data structures stored thereon. An example memory is further described with respect to FIG. 5.

The memory 124 may store an artificial intelligence model 126 and a detection algorithm 128. In some embodiments, the artificial intelligence model 126 may include a segmentation model configured to perform pixel-wise segmentation of video data. For example, the artificial intelligence model 126 may include a convolutional neural network having an encoder-decoder structure. In some embodiments, the artificial intelligence model 126 may be trained on surgical microscope video data from a wide variety of patient eyes captured with different surgical microscope platforms.

In some embodiments, the artificial intelligence model 126 may be configured to receive video data, including individual video frames from the camera 132 as input and to output masks identifying lens fragments and anatomical structures of the eye 140 in each video frame. A mask may be a two-dimensional image array of the same spatial dimensions as an input video frame in which each pixel may be assigned a value indicating either the presence or absence of a segmented structure. The anatomical structures identified by the artificial intelligence model 126 may include the pupil, the iris, the limbus, and the rhexis of the eye 140, among other structures of the eye 140. In some embodiments, the artificial intelligence model 126 may be configured to process the video data on a frame-by-frame basis in real time during the medical procedure.

In some embodiments, the detection algorithm 128 may be configured to analyze the masks produced by the artificial intelligence model 126 to track the identified lens fragments and to detect whether any identified lens fragments move outside a boundary of a pupil region of the eye 140. For example, the detection algorithm 128 may be configured to generate a velocity for each of the identified lens fragments. Alternately or additionally, the detection algorithm 128 may be configured to detect a disappearance of the identified lens fragments when the identified lens fragments do not follow a tracked trajectory. Alternately or additionally, the detection algorithm 128 may analyze a total volume of all tracked lens fragments across two or more video frames. In these and other embodiments, the detection algorithm 128 may detect whether any identified lens fragments move outside the boundary of the pupil region based on a total volume of all tracked lens fragments, the disappearance of the identified lens fragments, and/or the velocity of the identified lens fragments.

In some embodiments, the detection algorithm 128 may track each lens fragment across successive video frames using a multi-object tracking algorithm configured to associate lens fragments across consecutive video frames. For example, the detection algorithm 128 may include a combinatorial assignment algorithm configured to associate lens fragment in a current video frame with lens fragment from a prior video frame. In these and other embodiments, the detection algorithm 128 may also be configured to predict and update the estimated position and velocity of each tracked lens fragment across successive video frames. For example, the detection algorithm 128 may use a recursive linear state estimator to predict and update the estimated position and velocity of each tracked lens fragment. In some embodiments, the detection algorithm 128 may further include a sparse optical flow algorithm configured to estimate the displacement of image feature points associated with each lens fragment between consecutive video frames. The sparse optical flow algorithm may refine the position estimates produced by the recursive linear state estimator by computing actual pixel displacement of lens fragment contour points within a bounding box associated with each tracked lens fragment.

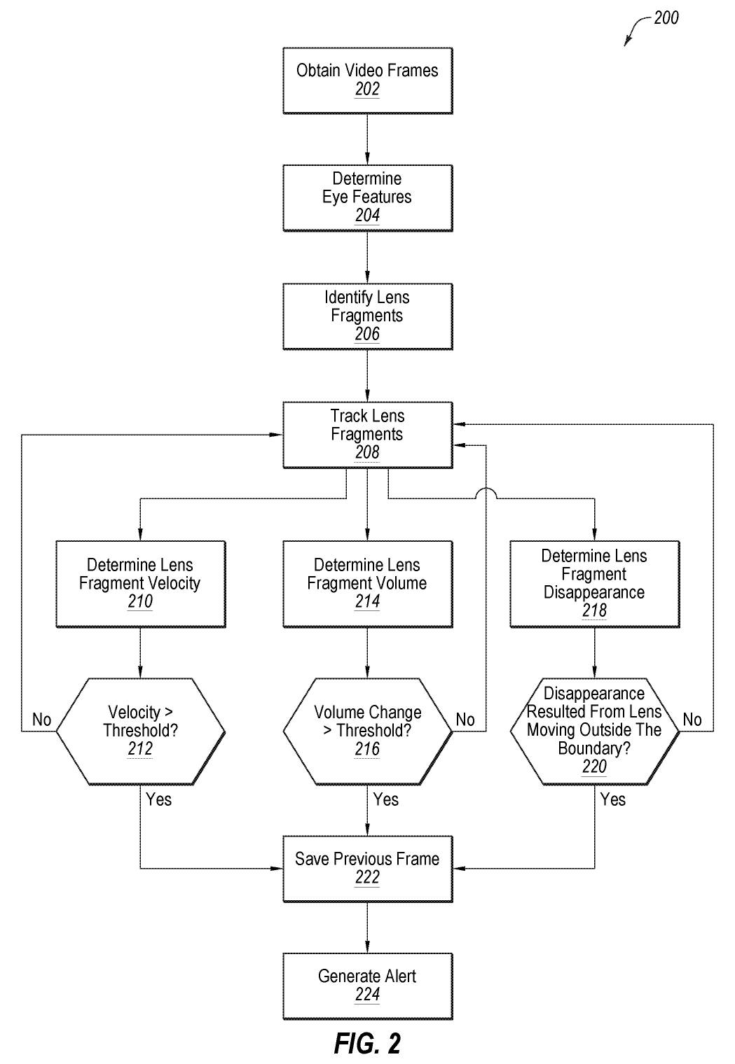

Further description of how the detection algorithm 128 may be configured to track the identified lens fragments and to detect whether any identified lens fragments move outside a boundary of a pupil region of the eye 140 is provided with respect to FIGS. 1B and 2.

In response to detecting lens fragments moving outside the boundary of the pupil region, the processing system 120 may generate an indication that lens fragments moved outside the boundary of the pupil region.

In some embodiments, the display 130 may be configured to present video, text, and other data as directed by the processor 122. In some embodiments, the display 130 may be a high-resolution surgical display monitor connected to the processing system 120. In these and other embodiments, the processing system 120 may be configured to obtain the video data from the camera 132 in real time as the surgeon performs the medical procedure on the eye 140 and present the video data on the display 130. As a result, the surgeon may view the display 130 as the surgeon performs the medical procedure.

In some embodiments, the display 130 may present further information regarding the lens fragments. For example, the display 130 may highlight the lens fragments in the video data presented by the display 130 by overlaying graphical elements in real time onto the video data. In some embodiments, the lens fragments may have different distinguishers, such as different colors, based on a location of the lens fragments. For example, lens fragments that are near the boundary of the pupil region may be a first color and lens fragments that are not near the boundary of the pupil region may be a second color. Alternately or additionally, lens fragments that are predicted to be more likely to cross the boundary of the pupil region may have a different color.

In some embodiments, the display 130 may present the indication generated by the processing system 120 that lens fragments moved outside the boundary of the pupil region. The indication may include a change in presentation of video data, such as an overlay over the video data. For example, the indication may be an overlay on the video data that includes text. The text may be a single word or phrase that indicates the lens fragments moved outside the boundary of the pupil region. Alternately or additionally, the indication may be some other change in the presentation of the video data. In some embodiments, the indication may include a visual indicator of an approximate location of the lens fragments detected outside the boundary of the pupil region. In some embodiments, the processing system 120 may automatically save a video frame immediately preceding detection of the one or more lens fragments moving outside the boundary of the pupil region. The saved video frame may allow a surgeon to review and visual the lens fragments that moved outside the boundary of the pupil region.

Modifications, additions, or omissions may be made to the environment 100 without departing from the scope of the present disclosure. For example, in some embodiments, the processing system 120 may be configured to receive aspiration pressure data from the aspiration probe 134. In these and other embodiments, the processing system 120 may correlate aspiration pressure spikes detected in the aspiration pressure data with lens fragment removal events and may compare a count of confirmed lens fragment removal events to a total tracked lens fragment count. In response to a discrepancy between the count of confirmed lens fragment removal events and the total tracked lens fragment count, the processing system 120 may generate an additional indication presented on the display 130.

FIG. 1B illustrates an example environment 150 for tracking lens fragments during a medical procedure on the eye 140. The environment 150 may be arranged in accordance with at least one embodiment described in the present disclosure. The environment 150 may include the eye 140 having an iris 160, a pupil 170, and a boundary 162 between the iris 160 and the pupil 170. In some embodiments, the boundary 162 may serve as a reference boundary for detecting whether any identified lens fragments 180a, 180b, 180c, 180d, and 180e, referred to collectively as lens fragments 180 move outside the pupil 170 and beneath the iris 160.

In some embodiments, the pupil 170 may define the circular or near-circular aperture through which the interior of the eye 140 may be visualized during the medical procedure. The iris 160 may surround the pupil 170 and may be a colored region of the eye 140. In some embodiments, the pupil 170 may have a center and a radius that may serve as reference landmarks for partitioning the pupil 170 into regions. The center and radius of the pupil 170 may be used by the detection algorithm 128 to define a tracking region 174 that includes a first region 176 and a second region 178.

In some embodiments, a portion of the pupil 170 may be defined as a non-tracking region 172 where the detection algorithm 128 may not actively track lens fragments. The detection algorithm 128 may not actively track lens fragments in the non-tracking region 172 because lens fragments located within the non-tracking region 172 may be at a relatively low risk of migrating beneath the iris 160. Note that not tracking lens fragments in the non-tracking region 172 may also reduce the computational load on the processing system 120 by limiting active tracking to regions of the pupil 170 where lens fragments present a greater risk of migrating beneath the iris 160.

In some embodiments, the non-tracking region 172 may be defined as the innermost concentric region within the pupil 170 and may be centered on the center of the pupil 170. In some embodiments, the non-tracking region 172 may be bounded by a radial distance from the center of the pupil 170 that satisfies a condition of being less than or equal to one-half of the radius of the pupil 170. Alternately or additionally, the radial distance from the center of the pupil 170 may be 0.25, 0.3, 0.4, 0.6, or some other radial distance.

As another example, the non-tracking region 172 may be defined as a dynamic periphery zone that is determined based on the detected position of the aspiration probe 134. For example, the dynamic periphery zone may be centered on the detected position of the aspiration probe 134. In these and other embodiments, the processing system 120 may be configured to detect a position of the aspiration probe 134 within the video data and to define the non-tracking region 172 accordingly. In some embodiments, a size of the dynamic periphery zone may be adjusted based on aspiration parameters of the aspiration probe 134, such that a higher aspiration force may correspond to a larger dynamic periphery zone. In these and other embodiments, lens fragments located within the non-tracking region 172 may be at a relatively low risk of migrating beneath the iris 160 because the lens fragments may be aspirated by the aspiration probe 134.

In some embodiments, the tracking region 174 may be defined as the annular region surrounding the non-tracking region 172 and extending outward to the boundary 162 of the pupil 170. In some embodiments, the tracking region 174 may be bounded radially between the non-tracking region 172 and the full radius of the pupil 170. In some embodiments, the tracking region 174 may be the region within which the detection algorithm 128 actively tracks lens fragments, such as the lens fragments 180. In these and other embodiments, lens fragments entering the tracking region 174 may be assigned track states by the detection algorithm 128 and may be analyzed for velocity, disappearance, and volume across successive video frames.

In some embodiments, the tracking region 174 may be further partitioned into the first region 176 and the second region 178. The first region 176 may correspond to a region, such as an annular band, adjacent to the non-tracking region 172. In some embodiments, the first region 176 may be defined by a radial distance from the center of the pupil 170 that satisfies a condition of being greater than one-half of the radius of the pupil 170 and less than or equal to three-quarters of the radius of the pupil 170.

In some embodiments, the second region 178 may correspond to the outermost region, such as an annular band, of the tracking region 174, that is proximal to the boundary 162 of the pupil 170. In some embodiments, the second region 178 may be defined by a radial distance from the center of the pupil 170 that satisfies a condition of being greater than three-quarters of the radius of the pupil 170 and less than or equal to the full radius of the pupil 170.

In some embodiments, the second region 178 may represent a highest-risk area within the pupil 170 for lens fragment migration beneath the iris 160. In these and other embodiments, lens fragments entering the second region 178 may be identified as having a higher likelihood of migrating beneath the iris 160. In some embodiments, the processing system 120 may distinguish lens fragments in the second region 178 from lens fragments in the first region 176. For example, the processing system 120 may visually distinguish lens fragments in the second region 178 from lens fragments in the first region 176.

FIG. 1B illustrates various lens fragments 180 as an example only of potential lens fragments. Other distributions and configurations of lens fragments may occur. As noted previously, the detection algorithm 128 may generate an estimated velocity for each of the lens fragments 180 as the lens fragments 180 move through the tracking region 174. In some embodiments, in response to a velocity of a lens fragment being larger than a threshold velocity when the lens fragment enters the tracking region 174 and/or enters the second region 178, the detection algorithm 128 may detect that the lens fragment may move outside the boundary 162 of the pupil 170. The threshold velocity may be based on a size of the tracking region 174, the second region 178, the first region 176, a size of the lens fragment, a surgery being performed, and/or preferences of a surgeon.

Furthermore, the detection algorithm 128 may also determine a total volume of all tracked lens fragments 180 across successive video frames. In these and other embodiments, the detection algorithm 128 may detect one or more lens fragments moving outside the boundary 162 of the pupil 170 based on changes in the total volume.

In these and other embodiments, the detection algorithm 128 may approximate the volume of each individual lens fragment. For example, the detection algorithm 128 may use a cylindrical geometric model in which each lens fragment may be treated as a right circular cylinder having a diameter d and a height h calculated from the video data. In some embodiments, the detection algorithm 128 may sum the estimated volumes of all tracked lens fragments 180 within the tracking region 174 across a sliding temporal window of successive video frames to produce a total volume value for each frame. The detection algorithm 128 may then compute a volume imbalance between frames that is compared to a volume threshold. The detection algorithm 128 may detect that one or more lens fragments have moved outside the boundary 162 of the pupil 170 in response to the volume imbalance exceeding the volume threshold. In some embodiments, the volume threshold may be based on a minimum detectable fragment volume that may correspond to the smallest fragment volume that the artificial intelligence model 126 may identify. In these and other embodiments, the volume threshold may be set at a value equal to or greater than the estimated volume of the smallest identified fragment. In some embodiments, the volume threshold may be further based on a surgery being performed and/or preferences of a surgeon.

Additionally, the detection algorithm 128 may also be configured to detect a disappearance event of a lens fragment based on a predicted location derived from a trajectory of the lens fragment across successive video frames. For example, the detection algorithm 128 may maintain a trajectory for each identified lens fragment 180 by recording positions of the lens fragments across multiple video frames. Based on the trajectory, the detection algorithm 128 may predict locations for the lens fragments in a subsequent video frame. In some embodiments, in response to the artificial intelligence model 126 failing to identify the lens fragment at or near the predicted location in the subsequent video frame, the detection algorithm 128 may determine a disappearance event for the lens fragment. In these and other embodiments, in response to disappearance events when the predicted location of the lens fragments fall at, near, or over the boundary 162 of the pupil 170, the disappearance event may result in the detection algorithm 128 detecting the disappeared lens fragment moved outside the boundary 162 of the pupil 170.

Alternately or additionally, when the predicted location of the lens fragment does not fall at, near, or over the boundary 162 of the pupil 170, the disappearance event may not result in the detection algorithm 128 detecting the disappeared lens fragment moving outside the boundary 162 of the pupil 170. In these and other embodiments, further analysis regarding co-located projected locations of lens fragment (where the lens fragments overlap and two lens fragments are identified as a single lens fragment), projected locations of lens fragment in the non-tracking region 172, among other factors may be performed by the detection algorithm 128 to determine whether the disappeared lens fragment moved outside the boundary 162 of the pupil 170.

Modifications, additions, or omissions may be made to the environment 150 without departing from the scope of the present disclosure. For example, in some embodiments, the number of regions within the tracking region 174 may differ from the two regions 176 and 178 illustrated in FIG. 1B. For example, the tracking region 174 may include a single region or three or more regions defined by different radial thresholds relative to the radius of the pupil 170. As another example, the non-tracking region 172 may be absent from the environment 150. For example, the detection algorithm 128 may actively track lens fragments across the entire area of the pupil 170.

FIG. 2 illustrates a flowchart of an example method 200 for tracking lens fragments during a medical procedure. The method 200 may be arranged in accordance with at least one embodiment described in the present disclosure. One or more operations of the method 200 may be performed, in some embodiments, by a device or system, such as the medical system 110 of FIG. 1A or another device or combination of devices. In these and other embodiments, the method 200 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 200 may begin at block 202, where video frames of the eye 140 may be obtained. In some embodiments, the video frames may be obtained from the camera 132 and may be obtained in real time as a surgeon performs the medical procedure on the eye 140.

At block 204, eye features of the eye 140 may be determined from the video frames. In some embodiments, the eye features may be determined by providing the video frames to the artificial intelligence model 126.

At block 206, lens fragments in a pupil region of the eye 140 may be identified. In some embodiments, the lens fragments may be identified by providing the video frames to the artificial intelligence model 126.

At block 208, the lens fragments 180 identified at block 206 may be tracked across successive video frames. In some embodiments, the detection algorithm 128 may track each of the lens fragments 180 across successive video frames using a multi-object tracking algorithm configured to associate lens fragments 180 across consecutive video frames.

In some embodiments, the tracking at block 208 may be performed on a frame-by-frame basis in real time during the medical procedure. In these and other embodiments, the tracking at block 208 may be performed for lens fragments 180 located within the tracking region 174, including the first region 176 and the second region 178, as described with respect to FIG. 1B.

From the tracking performed at block 208, the method 200 may proceed along three parallel analysis branches. A first branch may proceed to block 210, a second branch may proceed to block 214, and a third branch may proceed to block 216. In some embodiments, the three branches may be executed in parallel. Alternately or additionally, the three branches may be executed sequentially or in any order. Alternately or additionally, some combination of the branches may be performed. For example, only the first, only the second, or only the third branch may be performed. As another example, only the first and second branches, only the first and third branches, or only the second and third branches may be performed.

At block 210, a velocity of each tracked lens fragment 180 may be determined. In some embodiments, the detection algorithm 128 may compute the velocity of each tracked lens fragment 180 as the lens fragment 180 moves through the tracking region 174.

At block 212, a determination may be made as to whether the velocity of a tracked lens fragment 180 satisfies a threshold velocity. In response to the velocity not satisfying the threshold velocity, the method 200 may return to block 208 to continue tracking the lens fragments 180. In response to the velocity satisfying the threshold velocity, the method 200 may proceed to block 222.

At block 214, a volume of the lens fragments 180 may be determined. In some embodiments, the detection algorithm 128 may approximate a volume of each individual lens fragment 180. In these and other embodiments, the detection algorithm 128 may sum the estimated volumes of all tracked lens fragments 180 within the tracking region 174 to produce a total volume value for each video frame.

At block 216, a determination may be made as to whether a volume change of the lens fragments 180 satisfies a threshold. In some embodiments, the detection algorithm 128 may compute a volume imbalance between video frames and compare the volume imbalance to a volume threshold. In response to the volume change not satisfying the threshold, the method 200 may return to block 208 to continue tracking the lens fragments 180. In response to the volume change satisfying the threshold, the method 200 may proceed to block 222.

At block 218, a trajectory of each tracked lens fragment 180 may be determined. In some embodiments, the detection algorithm 128 may maintain a trajectory for each identified lens fragment 180 by recording positions of the lens fragments 180 across multiple video frames. In these and other embodiments, the trajectory may represent a time-ordered sequence of estimated centroid positions of each tracked lens fragment 180 across successive video frames. In some embodiments, the detection algorithm 128 may predict a location for each tracked lens fragment 180 in a subsequent video frame based on the trajectory.

At block 218, a determination may be made as to whether a lens fragment has disappeared. In some embodiments, the detection algorithm 128 may determine whether lens fragments have been identified at all the lens fragments predicted location.

At block 220, a determination may be made as to whether the disappearance event corresponds to detecting a lens fragment moved outside the boundary 162 of the pupil 170. For example, in response to the disappearance event occurring near, at, or over the boundary 162, it may be determined that a lens fragment moved outside the boundary 162 of the pupil 170. Alternately or additionally, in response to the disappearance event occurring in the second region 178, it may be determined that a lens fragment moved outside the boundary 162 of the pupil 170.

At block 222, a previous video frame may be saved. In some embodiments, the previous video frame may be saved in response to any one of the blocks 212, 216, and 220 detecting a lens fragment moved outside the boundary 162 of the pupil 170. Alternately or additionally, the previous video frame may be saved in response to any two of the blocks 212, 216, and 220 detecting a lens fragment moved outside the boundary 162 of the pupil 170. Alternately or additionally, the previous video frame may be saved in response to all three of the blocks 212, 216, and 220 detecting a lens fragment moved outside the boundary 162 of the pupil 170. Thus, the method 200 may determine that a lens fragment moved outside the boundary 162 of the pupil 170 based on any one of the blocks 212, 216, and 220, any two of the blocks 212, 216, and 220, or all three of the blocks 212, 216, and 220 detecting a lens fragment moved outside the boundary 162 of the pupil 170.

At block 224, an alert may be generated that one or more lens fragments moved outside the boundary 162 of the pupil 170. In these and other embodiments, the alert may be presented on the display 130.

Modifications, additions, or omissions may be made to the method 200 without departing from the scope of the present disclosure. For example, in some embodiments, the one or more of the three parallel analysis branches proceeding from block 208 may be removed.

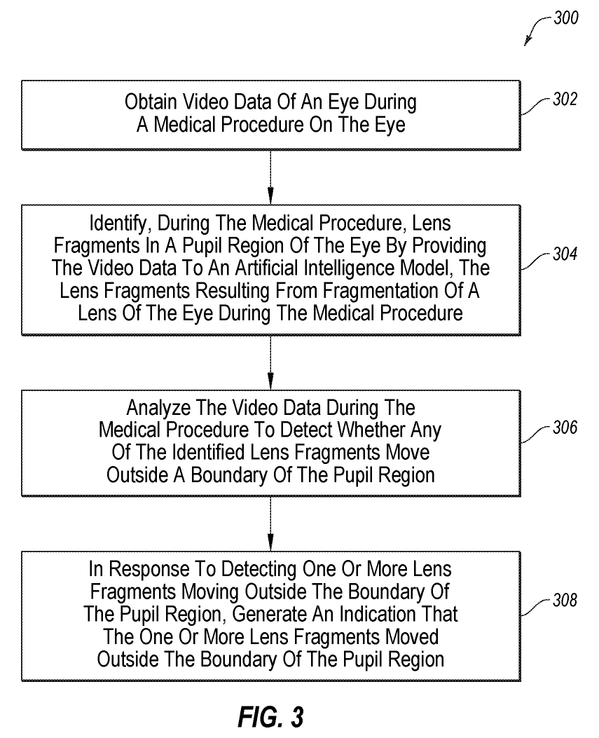

FIG. 3 illustrates a flowchart of an example method 300 for tracking lens material during a medical procedure on an eye. The method 300 may be arranged in accordance with at least one embodiment described in the present disclosure. One or more operations of the method 300 may be performed, in some embodiments, by a device or system, such as the medical system 110 of FIG. 1A or another device or combination of devices. In these and other embodiments, the method 300 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 300 may begin at block 302, where video data of an eye may be obtained during a medical procedure on the eye. In some embodiments, the video data may be obtained from the camera in real time as a surgeon performs the medical procedure on the eye. In some embodiments, the video data may include a continuous digital video stream comprising sequential video frames of the eye.

At block 304, multiple lens fragments in a pupil region of the eye may be identified during the medical procedure by providing the video data to the artificial intelligence model. In some embodiments, the plurality of lens fragments may result from fragmentation of a lens of the eye during the medical procedure.

At block 306, the video data may be analyzed during the medical procedure to detect whether any of the identified plurality of lens fragments move outside a boundary of the pupil region. In some embodiments, detecting whether any of the identified plurality of lens fragments move outside the boundary of the pupil region may include tracking, via the video data, one or more of the pluralities of lens fragments in a portion of the pupil region. In some embodiments, the portion of the pupil region may be an outer half of the pupil region.

In some embodiments, detecting whether any of the identified lens fragments move outside the boundary of the pupil region may include one or more of: tracking a volume of the plurality of lens fragments across two or more frames of the video data, tracking disappearance of the plurality of lens fragments from expected locations, and/or tracking velocities of the plurality of lens fragments as the plurality of lens fragments enter a portion of the pupil region.

At block 308, in response to detecting one or more lens fragments moving outside the boundary of the pupil region, an indication that the one or more lens fragments moved outside the boundary of the pupil region may be generated.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, in some embodiments, the method 300 may further include presenting the video data on a display during the medical procedure and presenting the indication on the display by overlaying the indication on the video data. In these and other embodiments, the indication may be presented to the surgeon in real time as the medical procedure proceeds.

In some embodiments, the method 300 may further include, in response to detecting the one or more lens fragments moving outside the boundary of the pupil region, providing a visual indicator of an approximate location of the one or more lens fragments outside the boundary of the pupil region. In these and other embodiments, the visual indicator may be overlaid on the video data presented on the display, providing the surgeon with spatially referenced information regarding the location of lens fragments that may have moved beneath the iris.

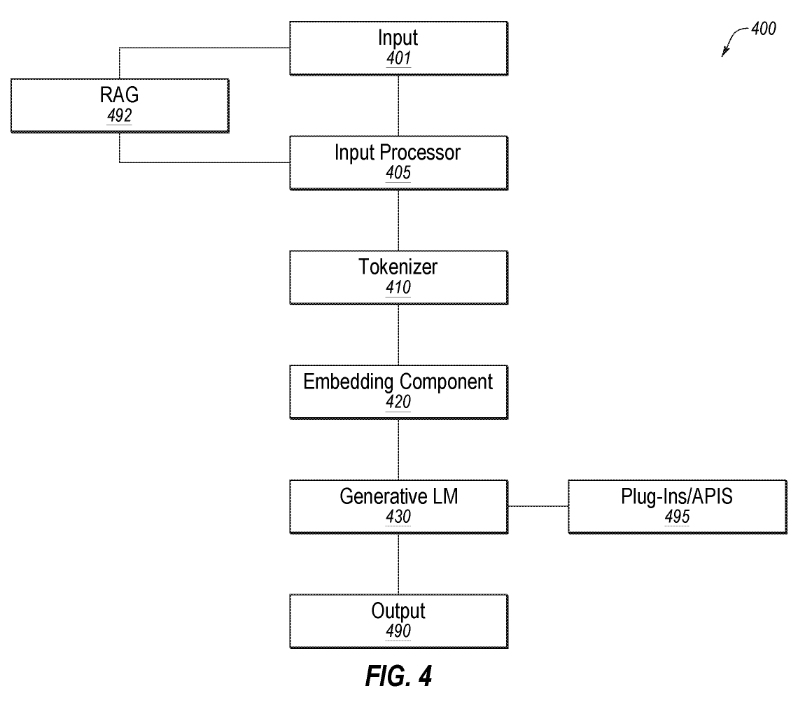

FIG. 4 is a block diagram of an example generative language model system 400 suitable for use in implementing at least some embodiments of the present disclosure. In the example illustrated in FIG. 4, the generative language model system 400 includes a retrieval augmented generation (RAG) component 492, an input processor 405, a tokenizer 410, an embedding component 420, plug-ins/APIs (Application Programming Interface) 495, and a generative language model (LM) 430 (which may include an LLM (large language model), a VLM (vision language model), a MMLM (multi-modal language model), a VLA (vision language action) model, etc.). The generative language model system 400 may be an example of a system that performs the AI models described with respect to FIG. 1.

At a high level, the input processor 405 may receive an input 401 comprising text and/or other types of input data (e.g., audio data, video data, frame data, sensor data), depending on the architecture of the generative LM 430 (e.g., LLM / LRM (Large Reasoning Model) / VLM / MMLM / etc.). Taking raw input text as an example, the input processor 405 may prepare raw input text in various ways. For example, the input processor 405 may perform various types of text filtering to remove noise (e.g., special characters, punctuation, HTML (Hypertext Markup Language) tags, stopwords, portions of a frame(s), portions of audio, etc.) from relevant textual content.

In some embodiments, a RAG component 492 (which may include one or more RAG models, and/or may be performed using the generative LM 430 itself) may be used to retrieve additional information to be used as part of the input 401 or prompt. RAG may be used to enhance the input to the LLM/LRM/VLM/MMLM/etc. with external knowledge, so that answers to specific questions or queries or requests are more relevant – such as in a case where specific knowledge is required. The RAG component 492 may fetch this additional information (e.g., grounding information, such as grounding text/frame/video/audio/USD /CAD/etc.) from one or more external sources, which can then be fed to the LLM/LRM/VLM/MMLM/etc. along with the prompt to improve accuracy of the responses or outputs of the model.

For example, in some embodiments, the input 401 may be generated using the query or input to the model (e.g., a question, a request, etc.) in addition to data retrieved using the RAG component 492. In some embodiments, the input processor 405 may analyze the input 401 and communicate with the RAG component 492 (or the RAG component 492 may be part of the input processor 405, in embodiments) in order to identify relevant text and/or other data to provide to the generative LM 430. The RAG component 492 may use various RAG techniques such as, naïve RAG, modular RAG techniques, and/or Graph RAG.

The tokenizer 410 may segment the (e.g., processed) text data into smaller units (tokens) for subsequent analysis and processing. The tokens may represent individual words, subwords, characters, portions of audio/video/frame/etc., depending on the implementation. For example, the tokenizer 410 may convert the (e.g., processed) text into a structured format according to tokenization schema being implemented in the particular embodiment.

The embedding component 420 may use any known embedding technique to transform discrete tokens into (e.g., dense, continuous vector) representations of semantic meaning. For example, the embedding component 420 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise. In some implementations in which the input 401 includes video data, the input processor 401 may extract frames or apply resizing to extracted frames, and the embedding component 420 may extract features such as optical flow embeddings or video embeddings and/or may encode temporal information or sequences of frames.

The generative LM 430 and/or other components of the generative LM system 400 may use different types of neural network architectures depending on the implementation. For example, transformer-based architectures such as those used in models like GPT (Generative Pre-trained Transformer) may be implemented and may include self-attention mechanisms. Some other non-limiting example architectures include transformers (e.g., encoder-decoder, decoder only, multi-modal), RNNs (Recurrent Neural Network), LSTMs (Long Short-Term Memory), fusion models, diffusion models, cross-modal embedding models that learn joint embedding spaces, graph neural networks (GNNs), hybrid architectures combining different types of architectures adversarial networks like generative adversarial networks or GANs (Generative Adversarial Network) or adversarial autoencoders (AAEs) for joint distribution learning, linear-time sequence modeling with selective state space modeling (SSM) architectures (e.g., Mamba LLM architectures), and/or others. As such, depending on the implementation and architecture, the embedding component 420 may apply an encoded representation of the input 401 to the generative LM 430, and the generative LM 430 may process the encoded representation of the input 401 to generate an output 490, which may include responsive text and/or other types of data.

As described herein, in some embodiments, the generative LM 430 may be configured to access or use - or capable of accessing or using - plug-ins/APIs 495 (which may include one or more plug-ins, application programming interfaces (APIs), databases, data stores, repositories, etc.). These and other architectures for LLMs / VLMs / MMLMs / VLAs / etc. described herein are meant simply as examples, and other suitable architectures may be implemented within the scope of the present disclosure.

FIG. 5 illustrates an example system 500 that may be used during transcription of communications. The system 500 may be arranged in accordance with at least one embodiment described in the present disclosure. The system 500 may include a processor 510, memory 512, a communication unit 516, a display 518, a user interface unit 520, and a peripheral device 522, which all may be communicatively coupled. In some embodiments, the system 500 may be part of any of the systems or devices described in this disclosure. For example, the system 500 may be part of the environment 100 of FIG. 1. Alternately or additionally, the system 500 may be used to perform one or more operations of the methods 200 and 300 of FIGS. 2 and 3.

Generally, the processor 510 may include any suitable special-purpose or general-purpose computer, computing entity, or processing device including various computer hardware or software modules and may be configured to execute instructions stored on any applicable computer-readable storage media. For example, the processor 510 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), quantum computer, or any other digital or analog circuitry configured to interpret and/or to execute program instructions and/or to process data.

Although illustrated as a single processor in FIG. 5, it is understood that the processor 510 may include any number of processors distributed across any number of networks or physical locations that are configured to perform individually or collectively any number of operations described herein. In some embodiments, the processor 510 may interpret and/or execute program instructions and/or process data stored in the memory 512. In some embodiments, the processor 510 may execute the program instructions stored in the memory 512.

For example, in some embodiments, the processor 510 may execute program instructions stored in the memory 512 that are related to selecting a transcription generation technique for a communication session such that the system 500 may perform or direct the performance of the operations associated therewith as directed by the instructions. In these and other embodiments, the instructions may be used to perform one or more operations of the methods described in this disclosure.

The memory 512 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 510.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 510 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.4d 1446 (Fed. Cir. 2007). Combinations of the above may also be included within the scope of computer-readable media.

The communication unit 516 may include any component, device, system, or combination thereof that is configured to transmit or receive information over a network. In some embodiments, the communication unit 516 may communicate with other devices at other locations, the same location, or even other components within the same system. For example, the communication unit 516 may include a modem, a network card (wireless or wired), an infrared communication device, a wireless communication device (such as an antenna), and/or chipset (such as a Bluetooth device, an 802.6 device (e.g., Metropolitan Area Network (MAN)), a WiFi device, a WiMax device, cellular communication facilities, etc.), and/or the like. The communication unit 516 may permit data to be exchanged with a network and/or any other devices or systems described in the present disclosure.

The display 518 may be configured as one or more displays, like an LCD (liquid crystal display), LED (light emitting diode), OLED (organic light emitting diode), Braille terminal, or other type of display. The display 518 may be configured to present video, text captions, user interfaces, and other data as directed by the processor 510. The user interface unit 520 may include any device to allow a user to interface with the system 500. For example, the user interface unit 520 may include a mouse, a track pad, a keyboard, joysticks, foot pedals, buttons, camera, and/or a touchscreen, among other devices. The user interface unit 520 may receive input from a user and provide the input to the processor 510. In some embodiments, the user interface unit 520 and the display 518 may be combined.

The peripheral devices 522 may include one or more devices. For example, the peripheral devices may include a microphone, a framer, and/or a speaker, among other peripheral devices. Modifications, additions, or omissions may be made to the system 500 without departing from the scope of the present disclosure. For example, in some embodiments, the system 500 may include any number of other components that may not be explicitly illustrated or described. Further, depending on certain implementations, the system 500 may not include one or more of the components illustrated and described.

As indicated above, the embodiments described herein may include the use of a special purpose or general-purpose computer (e.g., the processor 510 of FIG. 5) including various computer hardware or software modules, as discussed in greater detail below. Further, as indicated above, embodiments described herein may be implemented using computer-readable media (e.g., the memory 512 of FIG. 5) for carrying or having computer-executable instructions or data structures stored thereon.

Generally speaking, network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing systems(s) 500 of FIG. 5 – e.g., each device may include similar components, features, and/or functionality of the computing systems(s) 500. In addition, where backend devices (e.g., servers, NAS, etc.) are implemented, the backend devices may be included as part of a data center (such as, but not limited to, those described herein).

Components of a network environment may communicate with each other via a network(s), which may be wired, wireless, or both. The network may include multiple networks, or a network of networks. Compatible network environments may include one or more peer-to-peer network environments – in which case a server may not be included in a network environment – and one or more client-server network environments – in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may be private (e.g., limited to a single organization), may be public (e.g., available to many organizations), and/or a combination thereof (e.g., a hybrid cloud environment).

The various features illustrated in the drawings may be, but are not necessarily, drawn to scale. The illustrations presented in the present disclosure are not meant to be actual views of any particular apparatus (e.g., device, system, etc.) or method but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” among others).

Relative terms used in the present disclosure and especially in the appended claims (e.g., bodies of the appended claims) are generally intended as falling within manufacturing tolerances and/or within scope reasonably understood by a person of skill in the art. For example, if two components are identified as being the “same” size, there may be variations consistent with manufacturing variances. Terms describing “approximately,” “similar,” “substantially,” or other terms designating similarity may convey within ten percent of the comparative value. For example, two components that are approximately the same size would be understood to be of a size within ten percent of each other.

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more" to introduce claim recitations.

In addition, even if a specific number of an introduced claim recitation is explicitly recited, those skilled in the art will recognize that such recitation should be interpreted to mean at least the recited number (e.g., the bare recitation of "two recitations," without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of tracking lens material, the method comprising:
obtaining video data of an eye during a medical procedure on the eye;
identifying, during the medical procedure, a plurality of lens fragments in a pupil region of the eye by providing the video data to an artificial intelligence model, the plurality of lens fragments resulting from fragmentation of a lens of the eye during the medical procedure;
analyzing the video data during the medical procedure to detect whether any of the identified plurality of lens fragments move outside a boundary of the pupil region; and
in response to detecting one or more lens fragments of the plurality of lens fragments moving outside the boundary of the pupil region, generating an indication that the one or more lens fragments moved outside the boundary of the pupil region.

2. The method of claim 1, further comprising:
presenting, during the medical procedure, the video data on a display; and
presenting the indication on the display by overlaying the indication on the video data.

3. The method of claim 2, further comprising in response to detecting the one or more lens fragments moving outside the boundary of the pupil region, providing a visual indicator of an approximate location of the one or more lens fragments outside the boundary of the pupil region.

4. The method of claim 1, wherein detecting whether any of the identified plurality of lens fragments move outside the boundary of the pupil region includes tracking, via the video data, one or more of the plurality of lens fragments in a portion of the pupil region.

5. The method of claim 4, wherein the portion of the pupil region is an outer half of the pupil region.

6. The method of claim 4, wherein the portion of the pupil region is defined based on location of an aspiration probe within the boundary of the pupil region.

7. The method of claim 1, wherein detecting whether any of the identified plurality of lens fragments move outside the boundary of the pupil region includes one or more of:
tracking a volume of the plurality of lens fragments across two or more frames of the video data,
tracking disappearance of the plurality of lens fragments from expected locations, and
tracking velocities of the plurality of lens fragments as the plurality of lens fragments enter a portion of the pupil region.

8. The method of claim 1, wherein detecting whether any of the identified plurality of lens fragments move outside the boundary of the pupil region includes each of
tracking a volume of the plurality of lens fragments across two or more frames of the video data,
tracking disappearance of the plurality of lens fragments from expected locations, and
tracking velocities of the plurality of lens fragments as the plurality of lens fragments enter a portion of the pupil region.

9. The method of claim 8, wherein the one or more lens fragments are detected as moving outside the boundary of the pupil region in response to one or more of: the tracking the volume, the tracking the disappearance, and the tracking the velocities indicating the one or more lens fragments have moved outside the boundary of the pupil region.

10. A non-transitory computer-readable medium storing instructions that, when executed by one or more processors, cause the processors to perform the method of claim 1.

11. A system to track lens material, the system comprising:
a camera configured to generate video data of an eye during a medical procedure that fragments a lens of the eye;
one or more processors coupled to camera and configured to execute instructions to cause the system to perform operations, the operations comprising:
identifying, during the medical procedure, a plurality of lens fragments in a pupil region of the eye;
analyzing the video data during the medical procedure to detect whether any of the identified plurality of lens fragments move outside a boundary of the pupil region; and
in response to detecting one or more lens fragments of the plurality of lens fragments moving outside the boundary of the pupil region, generating an indication that the one or more lens fragments moved outside the boundary of the pupil region.

12. The system of claim 11, further comprising a display configured to present the video data, wherein the operations further comprise presenting the indication on the display.

13. The system of claim 12, wherein the operations further comprise in response to detecting the one or more lens fragments moving outside the boundary of the pupil region, presenting on the display a visual indicator of an approximate location of the one or more lens fragments outside the boundary of the pupil region overlaid on the video data.

14. The system of claim 11, wherein detecting whether any of the identified plurality of lens fragments move outside the boundary of the pupil region includes tracking, via the video data, one or more of the plurality of lens fragments in a portion of the pupil region.

15. The system of claim 14, further comprising an aspiration probe configured to aspirate the plurality of lens fragments, wherein the portion of the pupil region is defined based on location of the aspiration probe within the boundary of the pupil region.

16. The system of claim 11, wherein detecting whether any of the identified plurality of lens fragments move outside the boundary of the pupil region includes each of:
tracking a volume of the plurality of lens fragments across two or more frames of the video data,
tracking disappearance of the plurality of lens fragments from expected locations, and
tracking velocities of the plurality of lens fragments as the plurality of lens fragments enter a portion of the pupil region.

17. The system of claim 16, wherein the one or more lens fragments are detected as moving outside the boundary of the pupil region in response to one or more of: the tracking the volume, the tracking the disappearance, and the tracking the velocities indicating the one or more lens fragments have moved outside the boundary of the pupil region.

18. A medical system comprising:
a camera configured to generate video data of an eye during a medical procedure that fragments a lens of the eye;
an aspiration probe configured to aspirate a plurality of lens fragments resulting from the fragmentation of the lens;
one or more processors to:
identify, during the medical procedure, one or more of the plurality of lens fragments in a pupil region of the eye via the video data; and
analyze the video data during the medical procedure to detect whether any of the identified lens fragments move outside a boundary of the pupil region; and
a display coupled to the one or more processors and configured to present the video data and in response to detection of one or more identified lens fragments moving outside the boundary of the pupil region, present an indication that the one or more of the identified lens fragments moved outside the boundary of the pupil region.

19. The medical system of claim 18, wherein detecting whether any of the identified lens fragments move outside the boundary of the pupil region includes each of
tracking a volume of the identified lens fragments across two or more frames of the video data,
tracking disappearance of the plurality of lens fragments from expected locations, and
tracking velocities of the identified lens fragments as the identified lens fragments enter a portion of the pupil region.

20. The medical system of claim 19, wherein the one or more identified lens fragments are detected as moving outside the boundary of the pupil region in response to one or more of: the tracking the volume, the tracking the disappearance, and the tracking the velocities indicating the one or more identified lens fragments have moved outside the boundary of the pupil region.
